(19) United States
(12) Patent Application Publication
(10) Pub. No.: US 2009/0123405 A1
(43) Pub. Date: May 14, 2009

(54) Hair Shine and Styling Hold Composition And Methods Of Forming And Using Same

(75) Inventors: James Ziming Sun, Fountain Valley, CA (US); Gamze Sauzeat, Redondo Beach, CA (US); Angelika Huynh, Walnut, CA (US); Liwen Wu, San Diego, CA (US)

Correspondence Address: THE DIAL CORPORATION, 19001 N. Scottsdale Road, SCOTTSDALE, AZ, 85255, US

(73) Assignee: HENKEL CONSUMER GOODS, INC., Irvine, CA (US)

(21) Appl. No.: 12/097,746

(22) PCT Filed: Dec. 20, 2006
(86) PCT No.: PCT/US06/48936

§ 371 Date: Nov. 17, 2008

(30) Foreign Application Priority Data
Dec. 20, 2005 (US) 60597800

(51) Int. Cl. A61K 8/73; A61K 8/72; A61Q 5/06; A61K 8/81

(52) U.S. Cl. 424/70.13; 424/70.11; 424/70.16; 424/70.15

(57) ABSTRACT

A hair styling and shine composition and methods of using and forming the composition are disclosed. The composition includes about 0.1 wt % to about 60 wt % of a volatile silicone, about 0.1 wt % to about 20% wt % of hair fixative resin, and about 40 wt % to about 85% wt % of an organic solvent.

### CROSS-REFERENCE TO RELATED APPLICATIONS

This application is a U.S. National Phase filing under 35 U.S.C. 371 and claims priority to PCT Application No. PCT/US2006/048936, entitled “HAIR SHINE AND STYLING HOLD COMPOSITION AND METHODS OF FORMING AND USING SAME,” which was filed on Dec. 20, 2006, which application claims priority to U.S. Provisional Patent Application Ser. No. 60/597,800, entitled “HAIR SHINE AND STYLING HOLD SPRAY COMPOSITION,” which was filed Dec. 20, 2005, both of which are incorporated herein by reference.

### FIELD OF THE INVENTION

This invention generally relates to hair-care products, and more particularly, to compositions that provide shine and styling hold to hair.

### BACKGROUND OF THE INVENTION

Style-hold, hair-care products are often used to facilitate hair styling and to maintain a shape of styled hair. Such products often include resins or gums, which can be removed with water or cleansing of the hair, such that the styling-hold products can be removed during ordinary shampooing.

Similarly, various hair-care products often include materials such as silicones (e.g., organopolysiloxanes) that provide shine and leave hair feeling soft, smooth, and manageable. For example, shampoos for conditioning and enhancing hair shine, which include high molecular weight silicone with anionic surfactants, are discussed in U.S. Pat. No. 4,728,457 and U.S. Pat. No. 4,788,006. U.S. Pat. No. 6,709,648 discloses a hair conditioning product that includes a non-volatile silicone compound, having a molecular weight of from about 100,000 to about 1,500,000. The high molecular weight silicone product is combined with isoparaffin hydrocarbon and PEG-modified glycerides or PEG-modified glyceryl fatty acid esters to control hair frizz.

Both the shampoos and conditioners are water based and are typically applied to wet hair. Although these water-based products generally provide some benefit, such as manageability and smoothness, water in the products and on the hair tends to interact with the silicones to leave a relatively dull shine to hair.

Various aqueous spray compositions including silicones have also been developed. U.S. Pat. No. 5,176,898 and U.S. Pat. No. 5,294,437 disclose an aerosol spray including 0.005-0.5% of a volatile silicone selected from the group consisting of cyclomethicone or dimethicone copolyol dissolved in ethanol, a water and aerosol propellant mixture, and water soluble hair fixative resins. The water in hairspray dulls the hair shine and limits the composition's capability to carry silicones in the composition, which in turn interferes with the optimal performance of the composition.

U.S. Pat. Nos. 4,983,418 and 4,983,377 describe sprayable compositions, which include silicone gum emulsified by hydrophobically-modified quaternary ammonium treated clay in organic solvents with selected hair fixative resins. The clay-type emulsifier makes hair look dull, and the silicone gum tends to soften the hair significantly, which reduces the styling hold of the hair fixative resins.

Accordingly, an improved hair-care composition, which provides both hold and shine to hair and methods of using and forming the composition are desired.

### SUMMARY OF INVENTION

The present invention provides an improved hair-styling product and methods of using and forming the product. While the ways in which the present invention addresses the various drawbacks of the prior art are discussed in greater detail below, in general, the present invention provides a water-free, hair-care composition that provides both style retention and shine to hair.

In accordance with one embodiment of the invention, a composition for providing shine and style hold to hair includes about 0.1 to about 60% by weight of volatile silicones, from about 0.1 to about 20% by weight of hair fixative resins, and from about 40 to about 85% by weight of organic solvents (optionally including propellants). In accordance with various aspects of this embodiment, the composition includes a plurality of volatile silicones of different molecular weights. In accordance with further aspects, the composition includes additional components, such as neutralizing agents, propellants, fragrances, sunscreens, proteins, preservatives, and the like.

In accordance with another embodiment of the invention, a method of forming a hair-care product includes the step of admixing about 0.1 to about 60% by weight of volatile silicones, from about 0.1 to about 20% by weight of hair fixative resins, and from about 40 to about 85% by weight of organic solvents to form a composition.

In accordance with yet another embodiment of the invention, a method of using a hair-care product, which provides both styling hold and shine to hair, includes the steps of providing a composition that hair includes about 0.1 to about 60% by weight of a volatile silicones, from about 0.1 to about 20% by weight of hair fixative resins and from about 40 to about 85% by weight of organic solvents, and applying the composition to hair. In accordance with various aspects of this embodiment, the method includes spraying the composition onto substantially dry hair.

### DETAILS OF THE PREFERRED EMBODIMENTS

The following description is of exemplary embodiments only and is not intended to limit the scope, applicability or configuration of the invention in any way. Rather, the following description provides a convenient roadmap for implementing exemplary embodiments of the invention. Various changes to the described embodiments may be made in the function, steps, and relative amounts of the described ingredients, without departing from the scope of the invention as set forth. Additionally, though conveniently described herein in general terms of a new spray composition to deliver shine and styling hold for hair, the present invention may take on other product forms, including pourable liquids, spritzes, foams, creams, pastes, viscous gels, mousses, pomades, lacquers, non-aerosol pump-spray liquids, and the like. Furthermore, the compositions of the present invention may include adjuvants such as deodorants, fragrances, aromatherapy essences, herbs, infusions, antimicrobials, pediculicides, stimulants, essential oils, hair coloring, dying or tinting agents, anti-gray agents, sun-blockers, vitamins, antioxidants, surfactants and other wetting agents, Rogaine®, mica, silica, metal flakes or other glitter-effect materials, conditioning agents, anti-static agents, opacifiers, detackifying agents, penetrants, preservatives, emollients, and the like, and these compositions having additional consumer benefits (perceivable or otherwise) likewise fall within the ambit of the present invention.

In accordance with various embodiments of the invention, a composition for providing styling hold and shine to hair includes about 0.1 wt % to about 60 wt %, preferably about 20 to about 50 wt %, and more preferably about 35 to about 45 wt % of a volatile silicone; about 0.1 wt % to about 20 wt %, preferably about 1 to about 15 wt %, and more preferably about 2 to about 10 wt % of at least one hair fixative resin; and about 40 wt % to about 85% wt %, preferably about 40 to about 60 wt %, and more preferably about 45 to about 55 wt % of at least one organic solvent, optionally including propellants. All percents set forth herein are weight percents of the composition, unless otherwise indicated. In accordance with various aspects of this embodiment, the composition provides adequate hold, while still providing sufficient silicone(s) to provide desired shine and/or conditioning. Further, the composition is substantially free of water and thus is able to provide shine to hair, without the dulling effects of water.

Volatile silicones suitable for use with the composition of the present invention include, but are not limited to, cyclomethicone, a generic name of cyclic dimethyl polysiloxane compounds that conform to the formula [(CH3)2SiO]n, where n has a value between 3 and 7. The International Nomenclature of Cosmetic Ingredients (INCI) names for dimethyl polysiloxane compounds are: cyclotrisiloxane, when n is equal to 3; cyclotetrasiloxane, when n is equal to 4; cyclopentasiloxane, when n is equal to 5; cyclohexasiloxane, when n is equal to 6; and cycloheptasiloxane when n is equal to 7. Exemplary compositions include one of the cyclic dimethyl polysiloxane or the mixture of cyclic dimethyl polysiloxanes, or the mixture dimethicone and cyclomethicone.

Additional volatile silicones suitable for the present invention include, but are not limited to, dimethyl siloxane compounds, dimethicone, which conform to the formula (C2H6OSi)xC4H12Si, where x has a value between 2 to 5. The INCI names for these linear dimethyl siloxane compounds are: disiloxane, trisiloxane, tetrasiloxane and pentasiloxane. Further exemplary compositions include one or more dimethicone, and/or a mixture of dimethicone and cyclomethicone.

The composition may also include other lower molecular weight silicones to alter the evaporation time of the composition on hair. Suitable lower molecular weight silicones include, but are not limited to, caprylyl methicone, phenyl trimethicone, PEG/PPG-20/23 dimethicone, PEG/PPG-5/3 trisiloxane, PEG/PPG-23/6 dimethicone, PEG/PPG-18/18 dimethicone, PEG-12 dimethicone, PEG-8 dimethicone. When included, the composition includes from about 0.1 wt % to about 5.0 wt % lower molecular weight silicones.

Any hair fixative resin soluble and dispersible (determined at ambient conditions) in the mixture of volatile mixture of organic solvent(s) and silicones may be used in the composition. Exemplary hair fixative resins suitable for the composition include, but are not limit to, acrylates/acrylamide copolymer (e.g., Ultrahold Strong from BASF), aminoethylpropanediol-acrylates/acrylamide copolymer, vinyl caprolactam/vp/dimethylaminoethyl methacrylate copolymer (e.g., Amphomer LV-71 from National Starch), octylacrylamide/acrylates/butylaminoethyl methacrylate copolymer, polyvinylpyrrolidone (PVP), copolymers of (PVP) and methylmethacrylate, copolymers of PVP and vinylacetate (VA), polyvinyl alcohol (PVA), copolymers of PVA and crotonic acid, copolymers of PVA and maleic anhydride, hydroxypropyl cellulose, hydroxypropyl guar gum, sodium polystyrene sulfonate, PVP/ethylmethacrylate/methacrylic acid terpolymer, vinyl acetate/crotonic acid/vinyl neodecanoate copolymer, octylacrylamide/acrylates copolymer, monoethyl ester of poly(methyl vinyl ether-maleic acid), and octylacrylamide/acrylate/butylaminoethyl methacrylate copolymers, and Butyl Ester of PVM/MA Copolymer from Gantrez ES-225 (ISP). Mixtures of polymers may also be used. Exemplary mixtures include acrylates/acrylamide copolymer, aminoethylpropanediol-acrylates/acrylamide copolymer, vinyl caprolactam/vp/dimethylaminoethyl methacrylate copolymer, and octylacrylamide/acrylates/butylaminoethyl methacrylate copolymer.

A viscosity of a composition can be adjusted by manipulating an amount and/or type of resin in the composition. Resins such as Amphomer will contribute to the viscosity, increasing the resins may also increase the viscosity, especially if different grades of amphomer are used. (Amphomer LV=low viscosity, HV=high viscosity). Similar resins such as Gantrez ES-225 or ES-425 can also be used to adjust a viscosity of the composition.

With certain of the polymers it may be desirable to neutralize some acidic groups to promote solubility/dispersibility. Examples of suitable neutralizing agents include 2-amino-2-methyl-1,3-propanediol (AMPD), 2-amino-2-ethyl-1,3-propanediol (AEPD), 2-amino-2-methyl-1-propanol (AMP), 2-amino-1-butanol (AB), monoethanolamine (MEA), diethanolamine (DEA), triethanolamine (TEA), monoisopropanolamine (MIPA), diisopropanol-amine (DIPA), triisopropanolamine (TIPA), and dimethyl stearamine (DMS). When present, the composition includes from about 0.1% to about 15%, preferably from about 0.5% to about 10%, and more preferably, about 0.5% to about 2% neutralizing agent.

The present composition also includes a volatile carrier system—e.g., organic solvents or propellants, such as those conventionally used in resin hairspray formulations. Exemplary organic solvents include C1-C6 alcohols, for example, ethanol. The carrier system “carries” the silicone gum to the hair then volatilizes, leaving the particulate containing gum behind on the hair to provide hair conditioning and hairstyling hold. The carrier is present in the hairspray composition at from about 20% to about 70%, preferably from about 35% to about 60% by weight of the composition.

The present composition may be formulated in aerosol or non-aerosol forms. If an aerosol hairspray is desired, a propellant is included in the composition to expel the other materials from the container.

The propellant can be any liquefiable gas conventionally used for aerosol containers. Preferably, the density of the propellant or mixture thereof is such that pure propellant is not emitted from the container (e.g., less than 1). Examples of materials that are suitable for use as propellants include trichlorofluoromethane, dichlorodifluoromethane, dichlorotetrafluoroethane, monochlorodifluoromethane, trichlorotrifluoroethane, dimethylether, propane, n-butane and isobutane, used singly or admixed together. The hydrocarbons, particularly isobutane, used singly or admixed with other hydrocarbons are preferred due to their densities being less than 1.

An amount of the propellant gas present in the composition is governed by normal factors well known in the aerosol art. For hairsprays, the level of propellant is generally from about 3% to about 35%, preferably from about 5% to about 35% of the total composition. If a propellant such as dimethylether utilizes a vapor pressure suppressant (e.g., trichloroethane or dichloromethane) the amount of suppressant is included as part of the propellant. The compositions may also contain other optional components suitable for rendering such propellants more acceptable.

As noted above, the composition may optionally include fragrances, sunscreens (e.g., ethylhexyl methoxycinnamate and benzophenones) and proteins. Water normally gives adverse effects for hair shine and is thus not added to the compositions of the present invention.

The invention is further illustrated by the following exemplary formulations, which are intended as illustrations only and not as a limitation to the scope of the invention.

### Example 1

| INCI Name | Percentage (wt %) |
|---|---|
| Cyclomethicone | 35 |
| acrylates/acrylamide copolymer | 8.0 |
| Dimethicone | 2.0 |
| Aminomethyl Propanol | 1.0 |
| Ethylhexyl Methoxycinnamate (sunscreen) | 1.0 |
| Viscosity adjustor | q.s |
| Preservatives | q.s. |
| Fragrance | q.s. |
| Alcohol Denat. | To 100% |

The composition of Example 1 was sprayed on human hair and found to provide hold and shine.

### Example 2

| INCI Name | Percentage (%) |
|---|---|
| Cyclomethicone | 40 |
| Octylacrylamide/Acrylates/Butylaminoethyl | 5.0 |
| Methacrylate Copolymer | |
| Dimethicone | 2.0 |
| Caprylyl Methicone | 2.0 |
| Aminomethyl Propanol | 1.0 |
| Ethylhexyl Methoxycinnamate | 1.0 |
| Viscosity adjustor | q.s |
| Preservatives | q.s. |
| Fragrance | q.s. |
| Alcohol Denat. | To 100% |

The composition of Example 2 was sprayed on human hair and found to provide hold and shine.

### Example 3

| INCI Name | Percentage (%) |
|---|---|
| Cyclomethicone | 35 |
| aminoethylpropanediol-acrylates/acrylamide copolymer | 7.0 |
| Dimethicone | 2.0 |
| Caprylyl Methicone | 2.0 |
| Aminomethyl Propanol | 1.0 |
| Ethylhexyl Methoxycinnamate | 1.0 |
| Viscosity adjustor | q.s |
| Preservatives | q.s. |
| Fragrance | q.s. |
| Alcohol Denat. | To 100% |

The composition of Example 3 was sprayed on human hair and found to provide hold and shine.

### Example 4

| Trade name/INCI Name | Percentage (%) |
|---|---|
| KF 995 (Shin Etsu)/(Cyclopentasiloxane) | 35.0 |
| Amphomer LV-71(National Starch)/ | 5.0 |
| Octylacrylamide/Acrylates/Butylaminoethyl | |
| Methacrylate Copolymer | |
| Dow Corning 200 fluid, 1.5 cst (Dow Corning)/ | 2.0 |
| Dimethicone | |
| Silsoft 034 (OSI Specialties)/Caprylyl Methicone | 2.0 |
| AMP(Angus)/Aminomethyl Propanol | 1.0 |
| Uvinul MC-80 (BASF)/Ethylhexyl Methoxycinnamate | 1.0 |
| Viscosity adjustor | q.s |
| Preservatives | q.s. |
| Fragrance | q.s. |
| Alcohol Denat. | 21 |
| DME/Dimethylether | 33.0 |

The composition of Example 4 was sprayed on human hair and found to provide hold and shine.

The present invention has been described above with reference to a number of exemplary embodiments and examples. It should be appreciated that the particular embodiments shown and described herein are illustrative of the invention and its best mode and are not intended to limit in any way the scope of the invention as set forth in the claims. For example, although the composition and methods have been described in detail in connection with various hairsprays, the composition may alternatively be applied to hair using other means. Those skilled in the art having read this disclosure will recognize that changes and modifications may be made to the exemplary embodiments without departing from the scope of the present invention. These and other changes or modifications are intended to be included within the scope of the present invention, as expressed in the following claims.

We claim:

1. A hair care composition comprising;
about 20.0 wt % to about 60 wt % of a volatile silicone;
about 0.1 wt % to about 20% wt % of hair fixative resin;
about 40 wt % to about 85% wt % of organic solvent; and
wherein the composition is substantially water free and wherein the composition is configured to provide shine to hair.

2. The hair care composition of claim 1, wherein the organic solvent includes a propellant.

3. The hair care composition of claim 1, wherein the composition comprises about 35 wt % to about 45 wt % volatile silicone.

4. The hair care composition of claim 1, wherein the volatile silicone is selected from the group consisting of cyclomethicone, dimethicone, caprylyl methicone, phenyl trimethicone, PEG/PPG-20/23 dimethicone, PEG/PPG-5/3 trisiloxane, PEG/PPG-23/6 dimethicone, PEG/PPG-18/18 dimethicone, PEG-12 dimethicone, PEG-8 dimethicone, and mixtures thereof.

5. The hair care composition of claim 1, wherein the composition comprises about 0.1 wt % to about 5.0 wt % lower molecular weight silicones.

6. The hair care composition of claim 5, wherein the lower molecular weight silicones are selected from the group consisting of caprylyl methicone, phenyl trimethicone, PEG/PPG-20/23 dimethicone, PEG/PPG-5/3 trisiloxane, PEG/PPG-23/6 dimethicone, PEG/PPG-18/18 dimethicone, PEG-12 dimethicone, and PEG-8 dimethicone.

7. The hair care composition of claim 1, wherein the composition comprises a mixture of dimethicone and cyclomethicone.

8. The hair care composition of claim 1, wherein the hair fixative resin is selected from the group consisting of acrylates/acrylamide copolymer, aminoethylpropanediol-acrylates/acrylamide copolymer, vinyl caprolactam/vp/dimethylaminoethyl methacrylate copolymer, octylacrylamide/acrylates/butylaminoethyl methacrylate copolymer, polyvinylpyrrolidone (PVP), copolymers of (PVP) and methylmethacrylate, copolymers of PVP and vinylacetate (VA), polyvinyl alcohol (PVA), copolymers of PVA and crotonic acid, copolymers of PVA and maleic anhydride, hydroxypropyl cellulose, hydroxypropyl guar gum, sodium polystyrene sulfonate, PVP/ethylmethacrylate/methacrylic acid terpolymer, vinyl acetate/crotonic acid/vinyl neodecanoate copolymer, octylacrylamide/acrylates copolymer, monoethyl ester of poly(methyl vinyl ether-maleic acid), octylacrylamide/acrylate/butylaminoethyl methacrylate copolymers, and mixtures thereof.

9. The hair care composition of claim 1, wherein the hair fixative resin comprises a copolymer mixture selected from the group consisting of acrylates/acrylamide copolymer, aminoethylpropanediol-acrylates/acrylamide copolymer, vinyl caprolactam/vp/dimethylaminoethyl methacrylate copolymer, and octylacrylamide/acrylates/butylaminoethyl methacrylate copolymer.

10. The hair care composition of claim 1, further comprising a neutralizing agent.

11. The hair care composition of claim 10, wherein the neutralizing agent is selected from the group consisting of 2-amino-2-methyl-1,3-propanediol (AMPD), 2-amino-2-ethyl-1,3-propanediol (AEPD), 2-amino-2-methyl-1-propanol (AMP), 2-amino-1-butanol (AB), monoethanolamine (MEA), diethanolamine (DEA), triethanolamine (TEA), monoisopropanolamine (MIPA), diisopropanol-amine (DIPA), triisopropanolamine (TIPA), and dimethyl stearamine (DMS).

12. The hair care composition of claim 10, comprising about 0.1 wt % to about 15 wt % neutralizing agent.

13. The hair care composition of claim 1, wherein the organic solvent comprises a compound selected from the group consisting of C₁-C₆ alcohols.

14. The hair care composition of claim 1, wherein the organic solvent comprises ethanol.

15. The hair care composition of claim 1, wherein the composition comprises about 20 wt % to about 70 wt % of a carrier.

16. The hair care composition of claim 1, wherein the organic solvent includes a propellant selected from the group consisting of trichlorofluoromethane, dichlorodifluoromethane, dichlorotetrafluoroethane, monochlorodifluoromethane, trichlorotrifluoroethane, dimethylether, propane, n-butane and isobutene, and mixtures thereof.

17. The hair care composition of claim 16, wherein the propellant is present in an amount of about 3 wt % to about 35 wt %.

18. The hair care composition of claim 16, further comprising a vapor pressure suppressant.

19. A method of forming a composition for providing shine and styling hold to hair, the method comprising the steps of:
providing about 20 wt % to about 60 wt % of a volatile silicone;
providing about 0.1 wt % to about 20% wt % of hair fixative resin;
providing about 40 wt % to about 85% wt % of organic solvent; and
admixing the volatile silicone, fixative resin, and organic solvent to form a substantially water-free composition.

20. A method of styling and providing shine to hair, the method comprising the step of applying a composition comprising about 0.1 wt % to about 60 wt % of a volatile silicone; about 0.1 wt % to about 20% wt % of hair fixative resin; and about 40 wt % to about 85% wt % of organic solvent to hair.

21. The method of styling hair of claim 20, wherein the step of applying includes applying the composition to substantially dry hair.
